(19) United States
(12) Patent Application Publication
(10) Pub. No.: US 2021/0315726 A1
(43) Pub. Date: Oct. 14, 2021

(54) CONTACT PIECE AND URINE DISCHARGE DEVICE

(71) Applicant: Han-Chun Lin, Taichung City (TW)

(72) Inventor: Han-Chun Lin, Taichung City (TW)

(21) Appl. No.: 16/843,862

(22) Filed: Apr. 8, 2020

(51) Int. Cl. A61F 5/441; A61F 5/453; A61F 5/44

(52) U.S. Cl. CPC A61F 5/441; A61F 5/4408; A61F 5/453

(57) ABSTRACT

A contact piece and a urine discharge device are provided. The contact piece includes a ring having inlet and outlet openings in communication with each other and an elastic fitting membrane having an end coupled to the ring in a sealed condition and an opposite end forming a membrane surface to cover the inlet opening. The membrane surface is formed with a second through opening. The elastic fitting membrane is made of a material having an extremely high elastic expansion/contraction rate and waterproofness. The contact piece is combinable with a urine conducting piece to form a urine discharge device. The expandability of the elastic fitting membrane allows the contact piece to be fit to a male sexual organ and the elastic fitting membrane is in complete engagement with the sexual organ to prevent urine leaking, while the elastic fitting membrane applies a proper pressing force to the sexual organ.

### TECHNICAL FIELD OF THE INVENTION

The present invention relates generally to a contact piece and a urine discharge device, and more particularly to a urine discharge device and a replaceable contact piece for use by a user who suffers urinary incontinence, which help prevent urine leaking, does not make the sexual organ feel uncomfortable, feature fixing effect and structure simplicity and being easy to put on and take off.

### DESCRIPTION OF THE PRIOR ART

Urinary incontinence refers to urine leaking during heavily laughing, coughing, or any situation that the abdomen applies a force. Male urinary incontinence takes about 40% of the whole population. Although a diaper may be used to handle urinary incontinence, keeping urine in contact with human skin for an extended period would cause skin allergy or even ulceration. Taiwan Utility Model M382117, which discloses a urine collector, and Taiwan Patent 1520720, which discloses a urine discharge structure provide solutions that handles urine leaking of an incontinence patient without the need of wearing a diaper. However, such solutions have complicated structures and require tedious operations of wearing and may cause uncomfortableness of the male sexual organs. Further improvements are necessary.

### SUMMARY OF THE INVENTION

In view of the above, the present invention aims to provide a solution for the purposes of overcoming the shortcomings of the prior art devices concerning complication of structure, tediousness of wearing, and uncomfortableness of male sexual organ.

Thus, an objective of the present invention is to provide a contact piece, wherein through an arrangement that the contact piece is combined with an elastic fitting membrane, when the contact piece is fit to the sexual organ of a male user, the elastic fitting membrane is set in completely tight engagement with a contact surface of the sexual organ to achieve an effect of leaking prevention, and further, a pressing force that the elastic fitting membrane applies to the sexual organ is not overly large to make the sexual organ feel uncomfortable.

Another objective of the present invention is that the characteristics of the above contact piece allows combination with a separate urine conducting piece to form a urine discharge device, so that the urine discharge device is allowed, through the contact piece, to easily fit to the sexual organ of the male user to achieve effects of both preventing urine leaking and allowing the urine discharge device to attach to the sexual organ, allowing a user who suffers urine incontinence to have urine conducted away in a condition of lying on a bed to achieve an effect of easily putting on and taking off.

A further objective of the present invention is that connection with a connection band is available so that through the connection band being connectable with the urine discharge device and being fastened to a user's body, the urine discharge device of the present invention is operable not just for a user lying on a bed, but also usable for a user taking outdoor activity.

To achieve the above objectives, the present invention is such that the urine discharge device at least includes a contact piece and a urine conducting piece. The contact piece at least includes a ring and an elastic fitting membrane. The ring comprises an inlet opening and an outlet opening that are in communication with each other in a penetrating form, the ring defining a ring inner circumferential surface between the inlet opening and the outlet opening, the ring defining a ring outer circumferential surface on an outside thereof. The elastic fitting membrane is made of a material showing extremely high elastic expansion/contraction rate and waterproofness and has one end that is formed with a first through opening and in communication with the outside and an opposite end that is formed with a membrane surface that is extended in a radial direction. The membrane surface is formed with a second through opening. The first through opening and the second through opening are in communication with each other. An elastic fitting membrane outer circumferential surface is defined outside and between the first through opening and the second through opening. An elastic fitting membrane inner circumferential surface is defined inside and between the first through opening and a bottom surface. An end of the elastic fitting membrane associated with the first through opening is coupled to the ring to form a sealed condition with respect to the ring. The second through opening is in communication with the inlet opening of the ring. The second through opening is smaller than the inlet opening of the ring. The second through opening is in communication with the inlet opening and the outlet opening of the ring.

The urine conducting piece is hollow and comprises an interior space and has one side formed with a lead-in opening and an opposite side formed with a lead-out opening. The interior space is gradually reduced, at the side associated with the lead-out opening, to form a guide slope. The contact piece is coupled to and fixed to the lead-in opening to form a sealed condition. The lead-out opening is provided for a urine drain tube or a urine bag to connect therewith.

With the above structural arrangement, since the elastic fitting membrane of the contact piece has an extremely large elastic expansion/contraction rate, when the elastic fitting membrane is fit to a root of a male sexual organ, the opening will properly change the opening size thereof through expansion/contraction according to the size of the sexual organ so as to be in complete abutting engagement with the sexual organ to prevent urine leaking. The elastic fitting membrane does not apply an overly large pressing force to the sexual organ to cause discomfort of the sexual organ and may directly provide a moderate, proper attaching force to the sexual organ, so that when the urine discharge device is used on a user lying on a bed, there is no need to provide an addition measure to fix the urine discharge device, while the urine discharge device can be properly attach to and fix to the sexual organ and may move with a movement of the body of the user on the bed. For application of the urine discharge device to a user taking an outdoor activity, a connection band can be used to connect the urine discharge device to a waist or other part of the user so as to achieve an effect of fixing. Since the urine discharge device of the present invention requires no other garment for use in combination therewith, the urine discharge device of the present invention also features simple structure and easy wearing and removing. The present invention provides all the above-discussed advantages to protect a user suffering urine incontinence from being troubled by urine incontinence and make the sexual organ in a dry and comfortable condition.

### BRIEF DESCRIPTION OF THE DRAWINGS

FIG. 1 is a perspective view, in an exploded form, showing the present invention.

FIG. 2 is a perspective view, in an assembled form, showing the present invention.

FIG. 3 is a schematic view showing use of the present invention in a bed-lying condition.

FIG. 4 is a schematic view showing use of the present invention in an activity-taking condition.

FIGS. 5, 6, 7, and 8 are schematic views illustrating an operation of use of the present invention.

FIGS. 9, 10, and 11 are cross-sectional views illustrating an elastic fitting membrane of a contact piece having an opening that is made in different sizes.

FIGS. 12 and 13 are schematic views illustrating the contact piece fit to a penis to demonstrate conditions before and after erection of the sexual organ.

FIGS. 14 and 15 are schematic views illustrating the contact piece of the present invention fit to a penis to demonstrate an effect of leaking prevention can be maintained by means of extendable/contractable deformation of the elastic fitting membrane even when the sexual organ shifts in position.

FIG. 16 is a perspective view, in an exploded form, showing another example of the present invention.

FIG. 17 is a perspective view of FIG. 16 in an assembled form.

### DETAILED DESCRIPTION OF THE PREFERRED EMBODIMENT

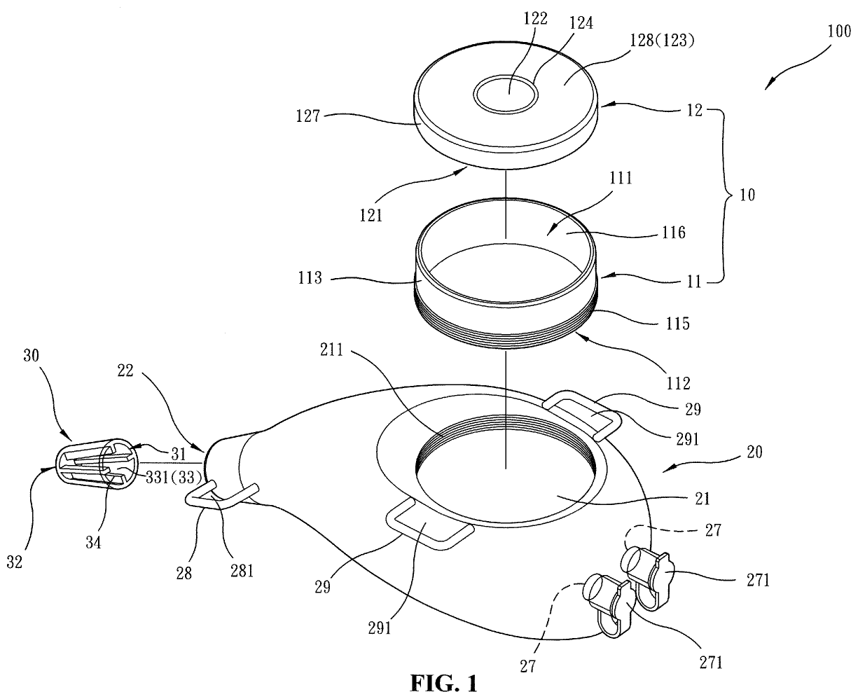

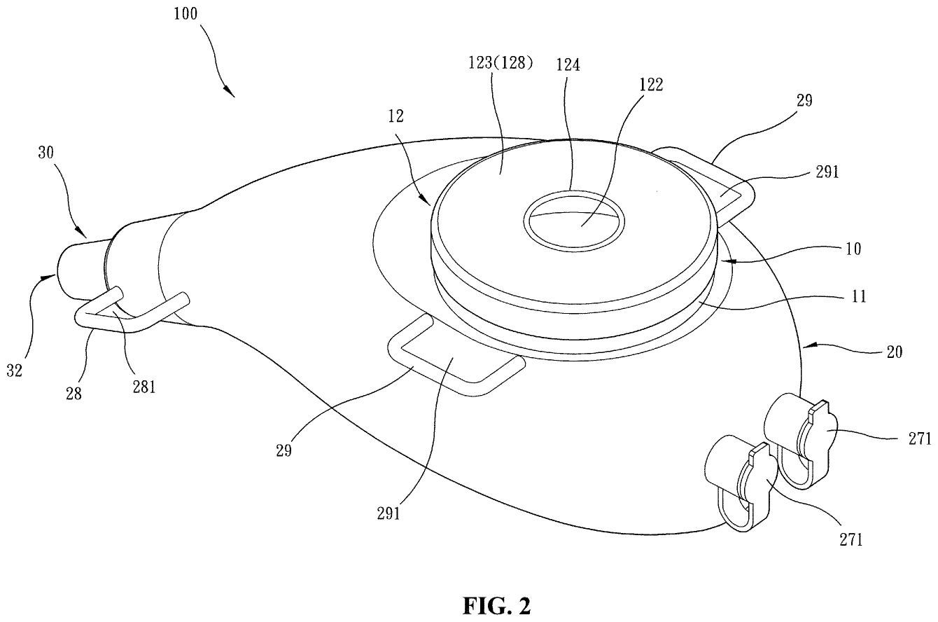

Referring to FIGS. 1, 2, and 5, a urine discharge device 100 according to an instant embodiment at least comprises a contact piece 10, a urine conducting piece 20, and 1 spreading piece 30.

The contact piece 10 includes a ring 11 and an elastic fitting membrane 12. The ring 11 has one side that is formed with an inlet opening 111 and an opposite side that is formed with an outlet opening 112. The inlet opening 111 and the outlet opening 112 are in communication with each other. The ring defines a ring inner circumferential surface 116 between the inlet opening 111 and the outlet opening 112, and also defines a ring outer circumferential surface 113 on an outside of the ring. The outer circumferential surface 113 is formed, on an end portion associated with the outlet opening 112, with an external thread 115. The elastic fitting membrane 12 has one end that is formed with a first through opening 121. The end of the elastic fitting membrane 12 associated with the first through opening 121 is formed in an integrally formed manner, to directly cover and fix to the ring outer circumferential surface 113 to provide a sealed condition. An opposite end of the elastic fitting membrane 12 is formed with a membrane surface 128 that is extended in a radial direction to cover the inlet opening 111 of the ring 11. The membrane surface 128 is formed, in a central portion thereof, with a second through opening 122. The second through opening 122 has a hole circumference that is formed with a lower circumferential rim 124, and a portion extending from the lower circumferential rim 124 to an elastic fitting membrane outer circumferential surface 127 constitute a radius shrinking flange 123. The first through opening 121 and the second through opening 122 are in communication with each other in a penetrating form. The elastic fitting membrane outer circumferential surface 127 is defined outside and between the first through opening 121 and the second through opening 122, and an elastic fitting membrane inner circumferential surface 125 is defined inside and between the first through opening 121 and the second through opening 122. The second through opening 122 is in communication with the inlet opening 111 and the outlet opening 112 of the ring 11. The second through opening 122 is smaller than the inlet opening 111 of the ring 11. The elastic fitting membrane 12 is made of a material showing extremely high elastic expansion/contraction rate and waterproofness, such as natural latex/rubber, natural latex/rubber added with additive(s), and aqueous polyurethane. Such materials have the properties of extremely high elastic expansion/contraction rate and waterproofness. Further, a preferred thickness of the elastic fitting membrane 12 is 0.04 mm, but can be thicker or thinner if desired. It is noted here that, in the instant embodiment, a way of combining the elastic fitting membrane 12 with the ring 11 is that the ring 11 is used in combination with a mold (not shown) positioned in the ring 11 and both are immersed in a liquid latex/rubber tank to have the latex/rubber attached to the ring outer circumferential surface 113 and also forming a membrane surface 128 on the inlet opening 111 of the ring 11 to thereby cover the inlet opening 111 of the ring 11, so that after the latex/rubber is cooled down and cured, secondary processing is carried out to cut the central portion of the membrane surface 128 to form the second through opening 122.

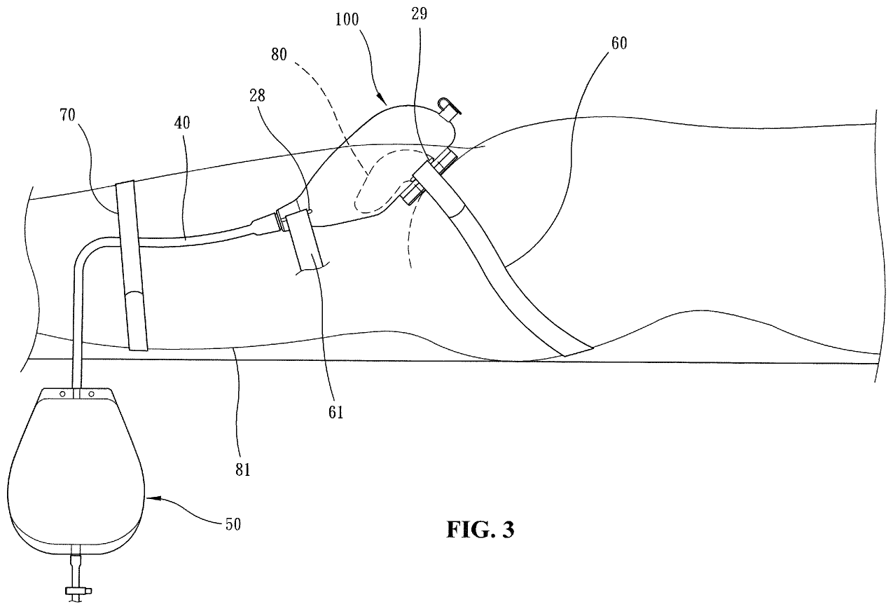

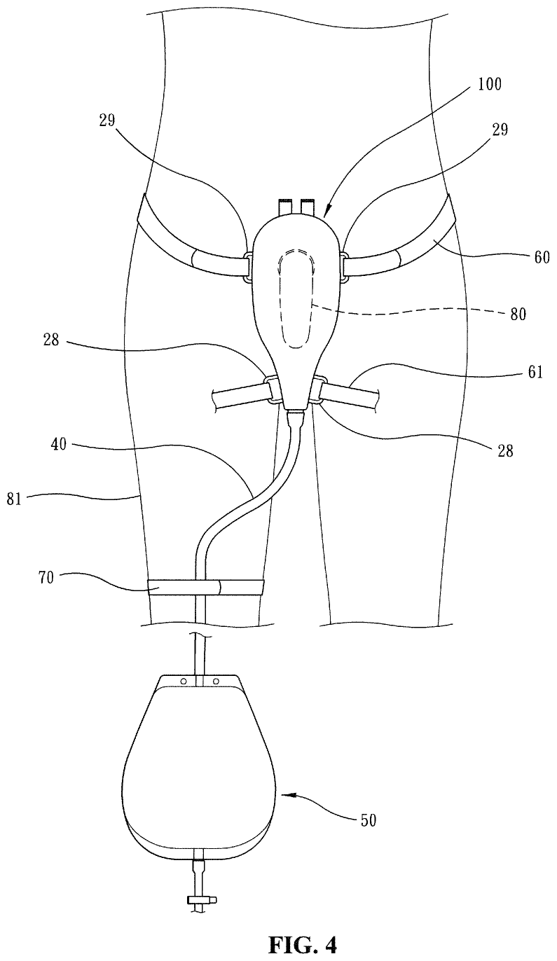

The urine conducting piece 20 is a hollow body, which includes an interior space 23. The urine conducting piece 20 has one side that is formed with a lead-in opening 21 in communication with the interior space 23. An opposite side of the urine conducting piece 20 is formed with a lead-out opening 22 in communication with the interior space 23. The lead-in opening 21 is formed with an internal thread 211 engageable with the external thread 115 of the ring 11 to provide a sealed condition therebetween. The interior space 23 is increasingly reduced from the side associated with the lead-in opening 21 toward the side associated with the lead-out opening 22. The interior space 23 is provided, in the side thereof associated with the lead-out opening 22, with an upper guide slope 25 and a lower guide slope 26 extending to the lead-out opening 22, so that urine is allowed to flow along the upper guide slope 25 and the lower guide slope 26 to the lead-out opening 22. The urine conducting piece 20 is provided, on each of two sides of the lead-in opening 21 and at a location close to each of two sides of the lead-out opening 22, with a mounting lug 29, 28 that is formed with a through hole 291, 281. The two mounting lugs 29(28) that are arranged at the same end allow two ends of a connection band 60, 61 to extend therethrough and fix thereto (see FIGS. 3 and 4). The urine conducting piece 20 includes two venting holes 27 at the side close to the lead-in opening 21 and in communication with the interior space 23. Each of the venting holes 27 is associated with a plug 271 to close or open the venting hole 27.

The spreading piece 30 is a tubular body having two ends that are open and in communication with each other in a penetrating form. One of the ends forms an ingress opening 31, and another end forms an egress opening 32. Defined between and extending from and in communication with the ingress opening 31 to the egress opening 32 is a flow channel 33 that is reduced in a direction toward the egress opening 32. The flow channel 33 has an inside surface that is formed with multiple barrier fences 34 that are raised therefrom and are spaced from each other so as to divide the flow channel 33 into multiple reduced flow channels 331, and is connected, with the end thereof associated with the ingress opening 31 to the lead-out opening 22 of the urine conducting piece 20 to be covered so as to form a sealed condition. The ingress opening 31 is set in communication with the interior space 23 of the urine conducting piece 20. The end associated with the egress opening 32 is also formed with a connecting section 35, and the connecting section 35 is separately connectable with and communicating with a urine drain tube 40.

Referring to FIG. 3, the urine discharge device 100 according to the present invention is coupled with a sexual organ 80 of a user lying on a bed in order to assist the user who suffers urine incontinence by conducting urine through the urine discharge device 100, a urine drain tube 40 connected to the urine discharge device 100, and a urine bag 50 connected to the urine drain tube 40 for collection of the urine. Often, the urine drain tube 40 is attached to a thigh 81 or a shank or the user by means of a fastening wire 70.

Referring to FIG. 4, the urine discharge device 100 according to the present invention is coupled with a sexual organ 80 of a user who is taking an activity for example outdoors and is used in combination with two connection bands 60 to be attached to a waist of the user. The connection bands 61 can be tied on the waist or other parts of the user to have the urine discharge device 100 securely attached and coupled to the sexual organ 80 of the user to assist the user who suffers urine incontinence by conducting urine through the urine discharge device 100, a urine drain tube 40 connected to the urine discharge device 100, and a urine bag 50 connected to the urine drain tube 40 for collection of the urine. Often, the urine drain tube 40 is attached to a thigh 81 or a shank or the user by means of a fastening wire 70.

Thus, the above provides an introduction to components and assembly thereof and uses of the urine discharge device 100 provided according to a preferred embodiment of the present invention, and in the following, the features of operation will be introduced.

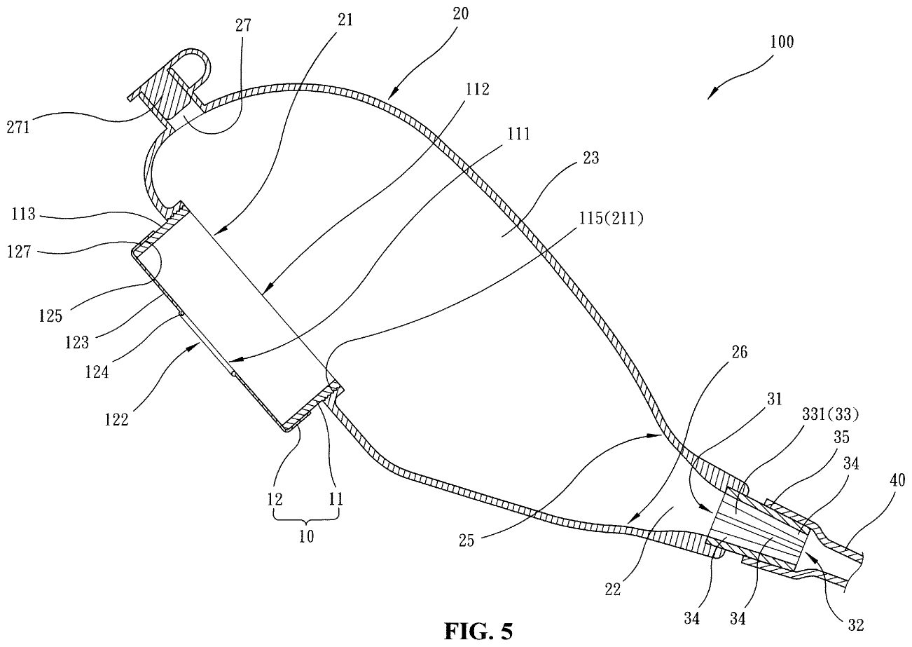

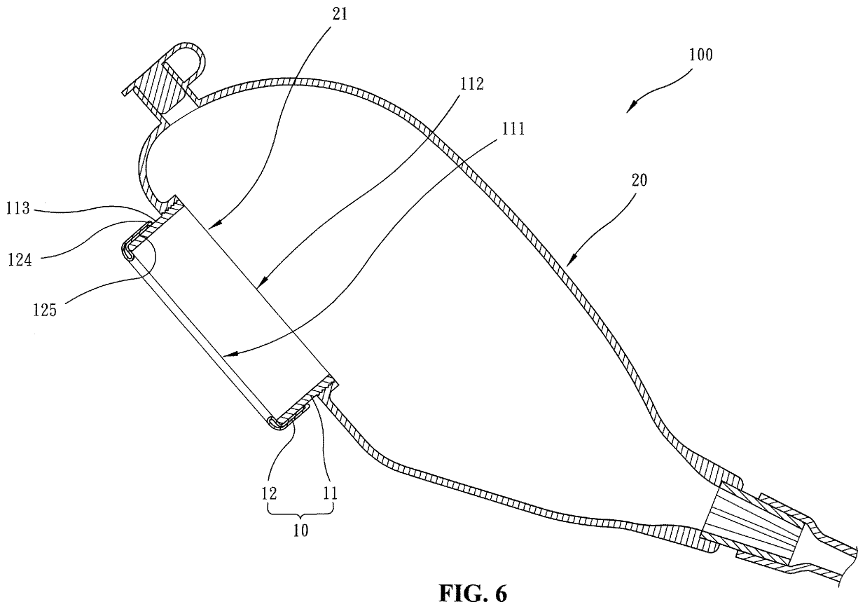

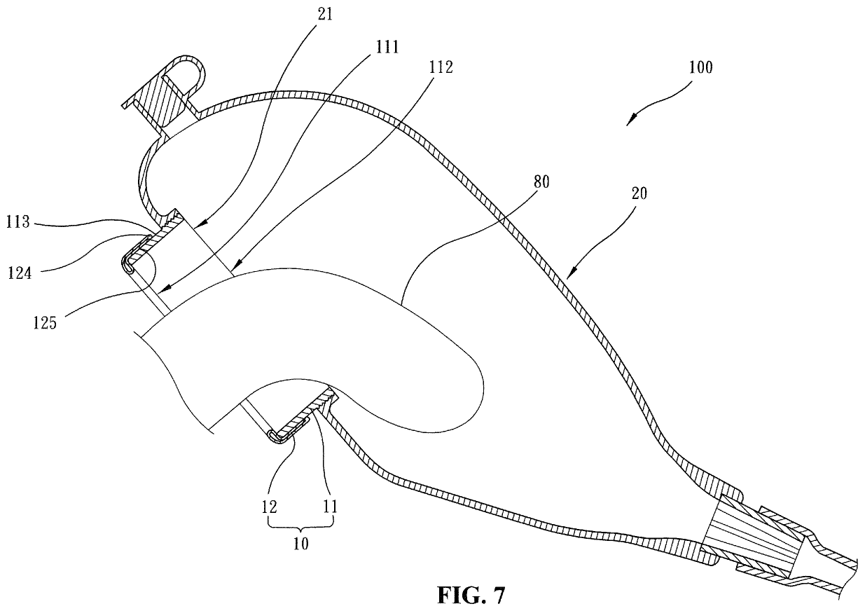

Referring to FIGS. 5-8, a user lying on a bed will be taken as an example for illustration. Before the elastic fitting membrane 12 of the urine discharge device 100 is applied to a sexual organ 80 of a user, the second through opening 122 of the elastic fitting membrane 12 must be first expanded in respect of the opening size thereof and the lower circumferential rim 124 and the radius shrinking flange 123 so expanded are fit over the ring outer circumferential surface 113, so that the inlet opening 111 of the ring 11 is no longer covered by the elastic fitting membrane 12 and the inlet opening 111 of the ring 11 is completely open and not blocked (see FIG. 6f). The sexual organ 80 of the user is then inserted, in sequence, through the inlet opening 111 and the outlet opening 112 of the ring 11 to be located in the interior space 23 of the urine conducting piece 20, and the inlet opening 111 of the ring 11 is set at a location adjacent to a root of the sexual organ 80. The sexual organ 80, in a condition of being not in erection and in a soft state, lays on an inner bottom surface of the urine conducting piece 20. Then, the lower circumferential rim 124 and the radius shrinking flange 123 of the elastic fitting membrane 12 are moved off the ring outer circumferential surface 113 to set back to the original position so that the lower circumferential rim 124 and the radius shrinking flange 123 of the elastic fitting membrane 12, due to elastic restoration power thereof, would resume the original condition, such that the lower circumferential rim 124 abuts a circumference of the sexual organ 80. Due to the extremely high elasticity property of the elastic fitting membrane 12, the sexual organ 80 would not feel uncomfortable, and further, the lower circumferential rim 124 applies a moderate or proper level of pressing force to the sexual organ 80 to thereby provide a coupling or combining force that is desired for fixing or holding the urine discharge device 100 to or on the sexual organ 800. Since the user is lying on a bed, the bottom of the urine conducting piece 20 is gently positioned on the scrotum of the user to support the weight of a major part of the urine discharge device 100. As such, the urine discharge device 100 is stably and securely coupled to and attached to the sexual organ 80 to move in unison with any position change or body turning of the user on the bed. In a practical usage, the connecting section 35 of the spreading piece 30 of the urine discharge device 100 is connected with a urine drain tube 40, and a distal end of the urine drain tube 40 is connected with a urine bag 50. Often, the urine drain tube 40 is fastened with a fastening wire 70 to have the urine drain tube 40 fixed to the thigh 81 or the shank, while the urine bag 50 is attached to an edge of the bed or any other suitable location. Thus, the weight of the urine drain tube 40 and the urine bag 50 does not stretch the urine discharge device 100 to cause detachment of the urine discharge device 100.

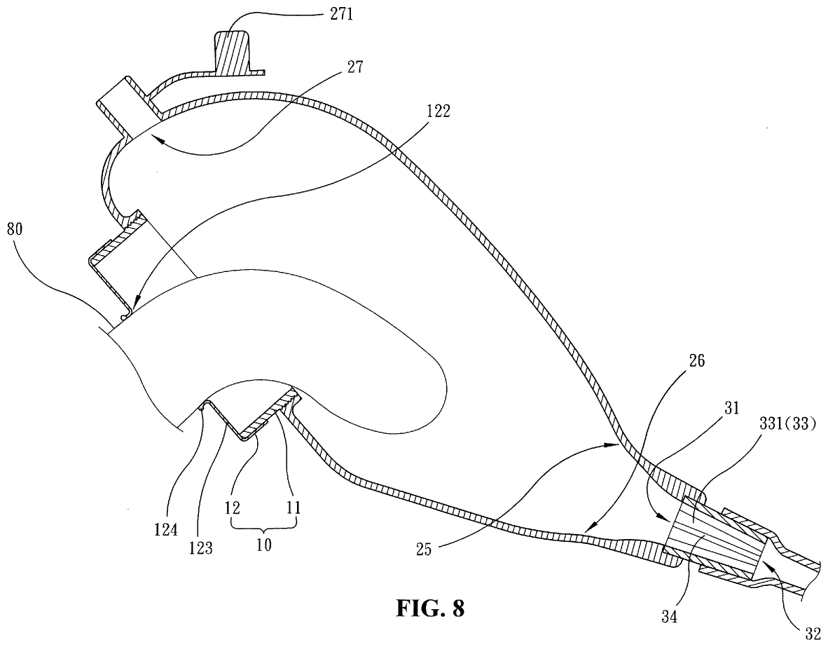

Referring further to FIG. 8, to use the urine discharge device 100 according to the instant embodiment, the plug 271 of the two venting holes 27 must be removed so that the two venting holes 27 are open for air ventilation to allow urine to fast flow along the upper guide slope 25 and the lower guide slope 26 to the spreading piece 30. The divided arrangement provided by the barrier fences 34 allows the urine to be conducted into each of the reduced flow channels 331, so that the urine can be drained quickly through the urine drain tube 40 without undesired accumulation in the spreading piece 30. Since the elastic fitting membrane 12 possesses a waterproofness property, an effect of preventing urine leaking between the lower circumferential rim 124 and the circumference of the sexual organ 80 in contact therewith may be achieved.

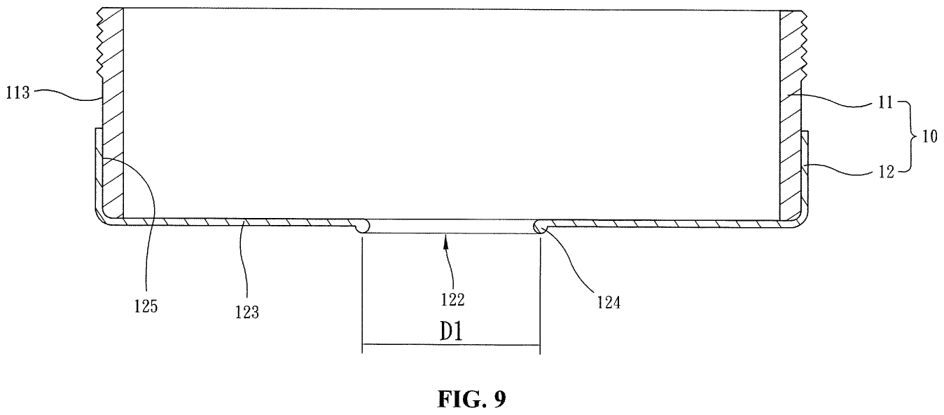

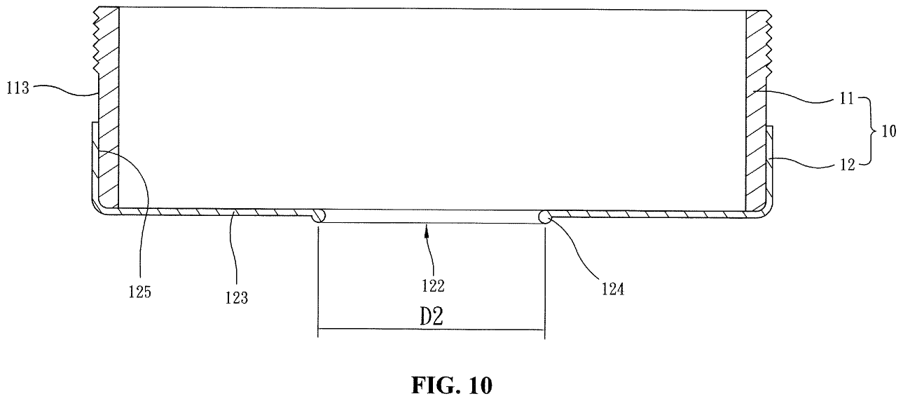

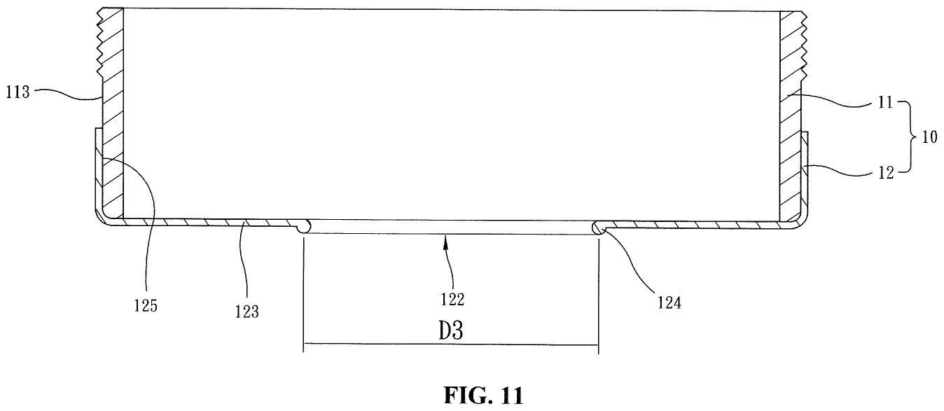

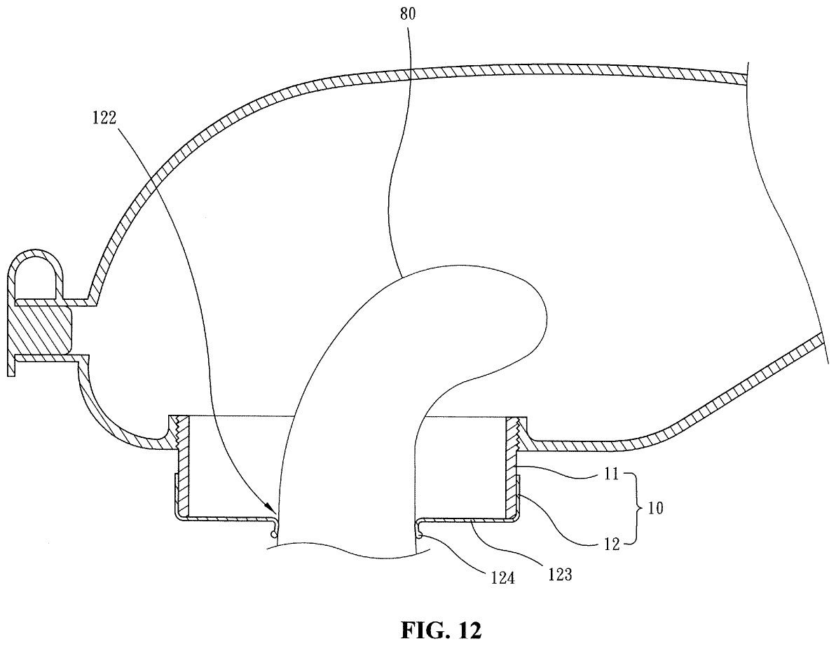

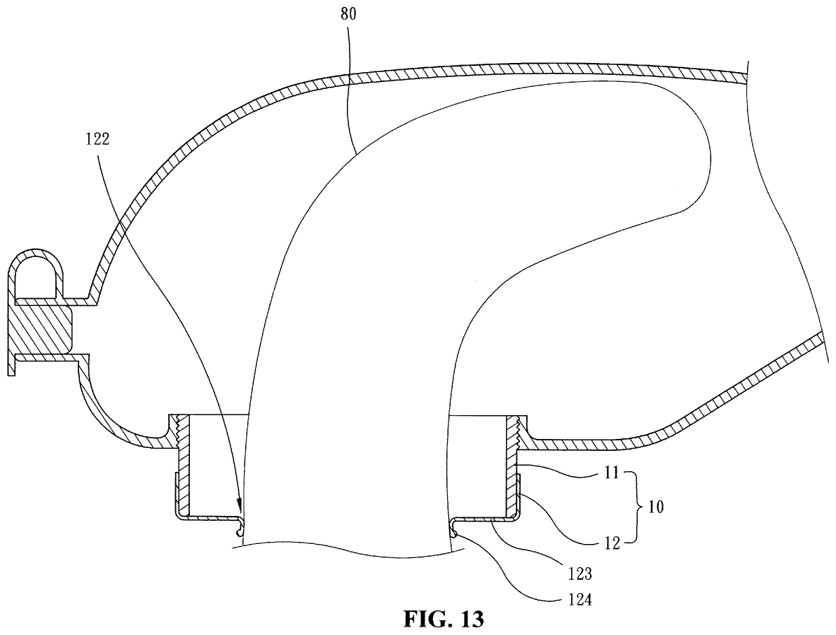

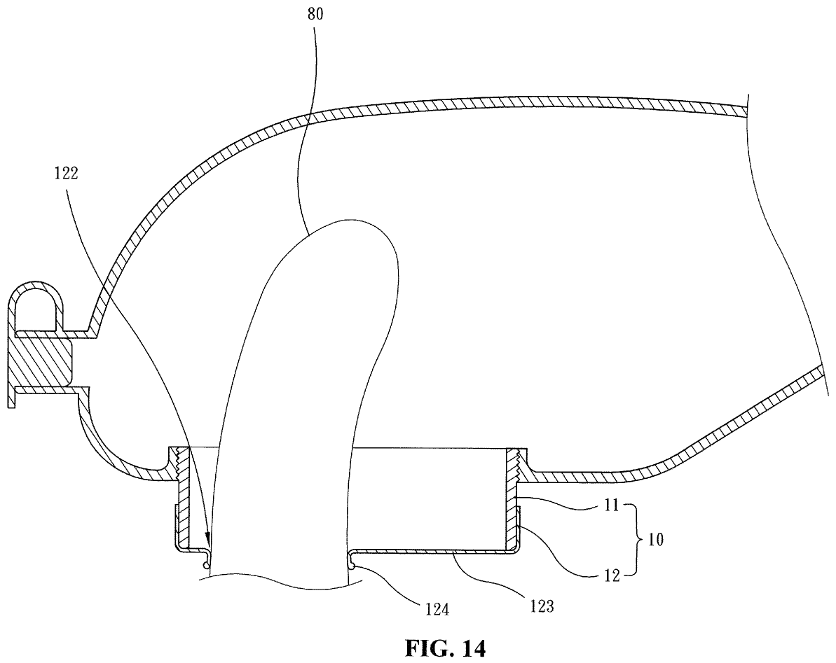

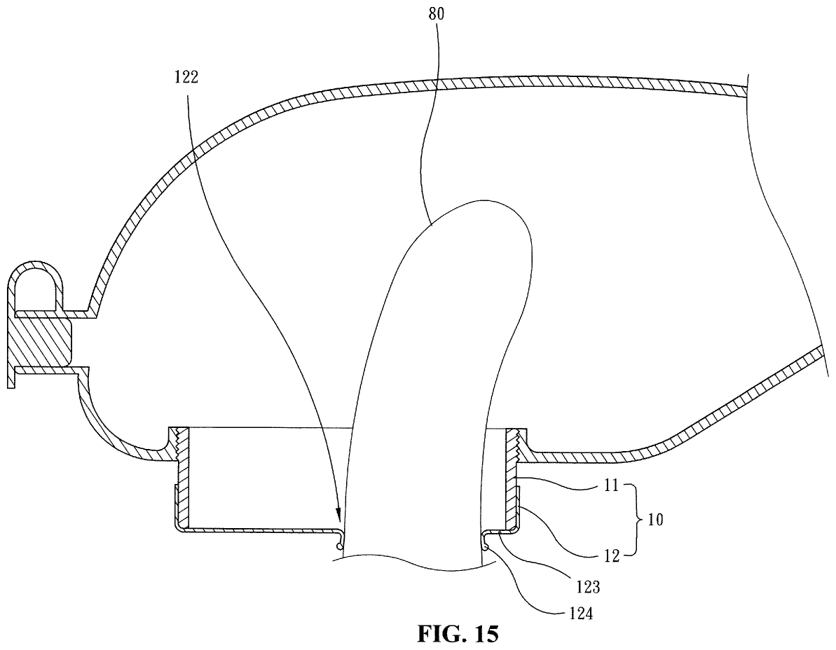

Referring further to FIGS. 9-11, the second through opening 122 of the elastic fitting membrane 12 of the contact piece 10 of the instant embodiment can be of various opening sizes, D1, D2, D3. Statistic data reveal that the sexual organs of the Asians normally have a maximum circumference of 11 millimeters (which is equivalent to a diameter of 3.5 millimeters) and a minimum circumference of 5.5 millimeters (which is equivalent to a diameter of 1.75 millimeters), an average value of circumference being 8.1 millimeters (which is equivalent to a diameter of 2.85 millimeters); and in an erected condition, the maximum circumference is 14.5 millimeters (which is equivalent to a diameter of 4.61 millimeters) and the minimum circumference is 8 millimeters (which is equivalent to a diameter of 2.55 millimeters), an average value of circumference being 11.1 millimeters (which is equivalent to a diameter of 3.53 millimeters). In the instant embodiment, a preferred size of the inner diameter of the ring 11 is taken as 5 millimeters and a preferred size of the second through opening 122 is set to 1.2, 1.6, or 2 millimeters. The various different sizes for the second through opening 122 so provided would suit the needs of the users for different sizes of sexual organ 80. Further referring to FIGS. 12 and 13, due to the extremely high elastic expansion/contraction rate of the elastic fitting membrane 12 that ensure easy expansion and contraction, when the sexual organ 80 is in a normal condition or an erected condition, the second through opening 122 of the elastic fitting membrane 12 could be automatically expanded or contracted and the lower circumferential rim can be kept in tight and abutting contact with the circumference of the sexual organ 80 so as to properly position and attach the urine discharge device 100 on and to the sexual organ 80 to prevent urine leading and not making the sexual organ 80 feel uncomfortable. However, in the instant embodiment, the sizes of the ring 11 and the second through opening 122 of the elastic fitting membrane 12 can be modified or designed according to the statistical data of the sexual organ size of different nations and different races. Further referring to FIGS. 14 and 15, when the contact piece 10 is being fit to the sexual organ 80 of a user, due to the property of expandability and contractibility of the radius shrinking flange 123, when the body of the user is being moved so that the sexual organ 80 is also caused to move inside the ring 11, the lower circumferential rim 124 may still keep in contact engagement with the sexual organ 80 to ensure an effect of preventing urine leaking.

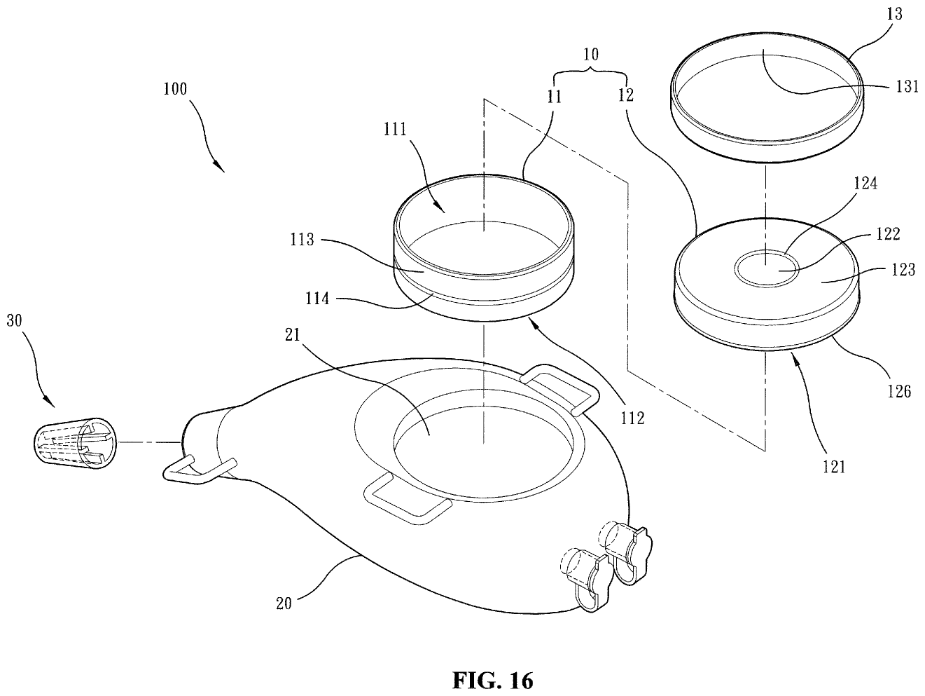

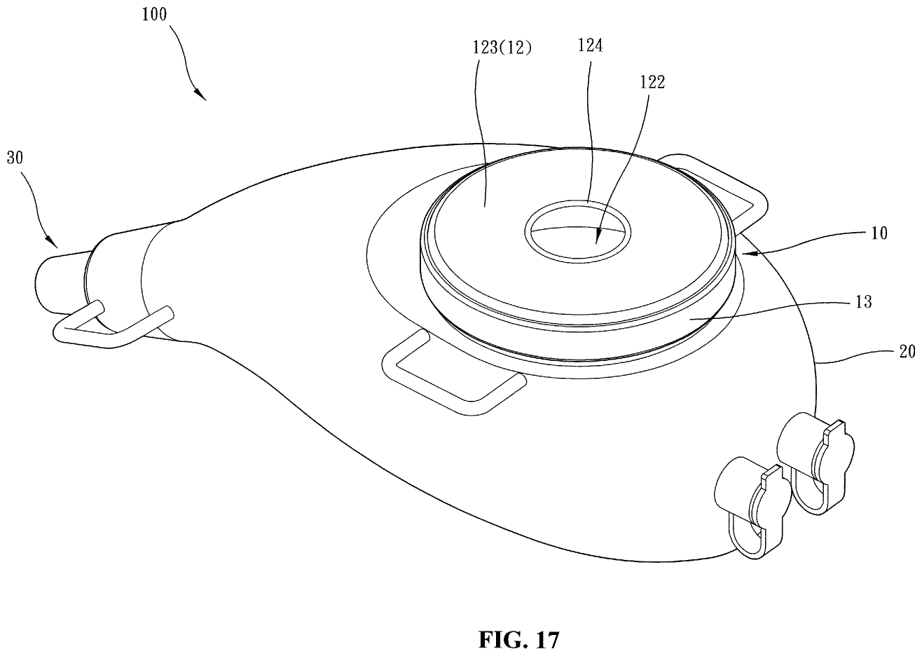

Referring to FIGS. 16 and 17, another example of the contact piece 10 according to the embodiment is shown and is different from the urine discharge device 100 of the first embodiment in that an arrangement of fixing through assembly of the elastic fitting membrane 12 and the ring 11 is adopted, wherein an end of the elastic fitting membrane 12 opposite to the second through opening 122 is formed with a first through opening 121 that is in communication with the outside. The first through opening 121 is formed, along a circumference thereof, with an upper circumferential rim 126, and the ring outer circumferential surface 113 is formed with a circumferential groove 114. The first through opening 121 of the elastic fitting membrane 12 is fit over the ring outer circumferential surface 113, and the upper circumferential rim 126 is set into engagement with and fitting into the circumferential groove 114. The elastic fitting membrane inner circumferential surface 125 is set in abutting and tight engagement with the ring outer circumferential surface 113, and the end of the elastic fitting membrane 12 associated with the second through opening 122 covers the inlet opening 111 of the ring 11. The end of the elastic fitting membrane 12 associated with the inlet opening 111 has an axial length that is slightly projecting beyond the inlet opening 111 of the ring 11, and finally, the outer fitting loop 13 that has two open ends in a penetrating form is fit over the ring outer circumferential surface 113 to be set at a location corresponding to the fitting site between the upper circumferential rim 126 and the circumferential groove 114, so that by means of the sizes of the ring outer circumferential surface 113 and the outer fitting loop inner circumferential surface 131 being close to each other, the outer fitting loop 13 and the ring 11 and the elastic fitting membrane 12 can be tightly fixed together to achieve an effect of preventing leaking. Further, the end of the ring 11 associated with the outlet opening 112 is covered by the lead-in opening 21 of the urine conducting piece 20 as being integrally combined together without being detached from each other, this also achieving the effect of positioning the urine discharge device 100 on the sexual organ, preventing urine leaking, and not making the sexual organ feel uncomfortable.

I claim:

1. A contact piece, at least comprising a ring and an elastic fitting membrane;
the ring comprising an inlet opening and an outlet opening that are in communication with each other in a penetrating form, the ring defining a ring inner circumferential surface between the inlet opening and the outlet opening, the ring defining a ring outer circumferential surface on an outside thereof;
the elastic fitting membrane having a property of elastic expansion/contraction and waterproofness and having one end that is formed with a first through opening and in communication with the outside and an opposite end that is formed with a membrane surface that is extended in a radial direction, the membrane surface being formed with a second through opening, the first through opening and the second through opening being in communication with each other, an elastic fitting membrane outer circumferential surface being defined outside and between the first through opening and the second through opening, an elastic fitting membrane inner circumferential surface being defined inside and between the first through opening and the second through opening, an end of the elastic fitting membrane associated with the first through opening being coupled to the ring to form a sealed condition with respect to the ring, the second through opening being in communication with the inlet opening of the ring, the second through opening being smaller than the inlet opening of the ring, the second through opening being in communication with the inlet opening and the outlet opening of the ring.

2. The contact piece according to claim 1, further comprising an outer fitting loop having two ends in communication with each other in a penetrating form, the ring outer circumferential surface being formed with a circumferential groove, the first through opening of the elastic fitting membrane being formed with an upper circumferential rim, the upper circumferential rim being engageable with and fit in the circumferential groove, the outer fitting loop being fit over an engagement site of the upper circumferential rim and the circumferential groove, the outer fitting loop inner circumferential surface and the ring outer circumferential surface having sizes that are close to each other, so that the ring, the elastic fitting membrane, and the outer fitting loop are tightly fit to and combine with each other.

3. The contact piece according to claim 1, wherein the coupling between the end of the elastic fitting membrane associated with the first through opening and the ring is achieved in an integrally formed manner to be directly covered and combined together.

4. The contact piece according to claim 1, wherein the elastic fitting membrane is made of a material of one of natural latex/rubber, natural latex/rubber added with an additive, and aqueous polyurethane.

5. The contact piece according to claim 1, wherein the second through opening of the elastic fitting membrane is formed with a lower circumferential rim, a portion from the lower circumferential rim to the elastic fitting membrane outer circumferential surface forming a radius shrinking flange.

6. The contact piece according to claim 2, wherein the second through opening of the elastic fitting membrane is formed with a lower circumferential rim, a portion from the lower circumferential rim to the elastic fitting membrane outer circumferential surface forming a radius shrinking flange.

7. A urine discharge device, at least comprising a contact piece and a urine conducting piece;
the contact piece comprising a ring and an elastic fitting membrane; the ring comprising an inlet opening and an outlet opening that are in communication with each other, the ring defining a ring inner circumferential surface between the inlet opening and the outlet opening, the ring defining a ring outer circumferential surface on an outside thereof; the elastic fitting membrane having a property of elastic expansion/contraction and waterproofness and having one end that is formed with a first through opening and in communication with the outside and an opposite end that is formed with a membrane surface that is extended in a radial direction, the membrane surface being formed with a second through opening, the first through opening and the second through opening being in communication with each other, an elastic fitting membrane outer circumferential surface being defined outside and between the first through opening and the second through opening, an elastic fitting membrane inner circumferential surface being defined inside and between the first through opening and the second through hole, an end of the elastic fitting membrane associated with the first through opening being coupled to the ring to form a sealed condition with respect to the ring, the second through opening being in communication with the inlet opening of the ring, the second through opening being smaller than the inlet opening of the ring, the second through opening being in communication with the inlet opening and the outlet opening of the ring;
the urine conducting piece being such that the urine conducting piece comprises a hollow body, which comprises an interior space and has one side formed with a lead-in opening in communication with the interior space and an opposite side formed with a lead-out opening in communication with the interior space, the lead-in opening being coupled with the ring of the contact piece to form a sealed condition, the interior space being provided, in the side thereof associated with the lead-out opening, with an upper guide slope and a lower guide slope that extend to the lead-out opening.

8. The urine discharge device according to claim 7, further comprising a spreading piece, the spreading piece being a tubular body having two open ends in a penetrating form, one of the ends forming an ingress opening, another one of the ends forming an egress opening, a flow channel being formed from the ingress opening to the egress opening so as to be in communication with each other and being arranged to reduce in a direction toward the egress opening, the flow channel having an inside surface that is formed with multiple barrier fences that are raised therefrom and are spaced from each other so as to divide the flow channel into multiple reduced flow channels, and is connected, with the end thereof associated with the ingress opening to the lead-out opening of the urine conducting piece to be covered so as to form a sealed condition, the ingress opening being set in communication with the interior space of the urine conducting piece, the end associated with the egress opening being also formed as a connecting section.

9. The urine discharge device according to claim 7, wherein the urine conducting piece comprises two venting holes in communication with the interior space, each of the venting holes being provided with a plug for closing or opening the venting hole.

10. The urine discharge device according to claim 7, at least further comprising a connection band, the urine conducting piece having two sides each provided with a mounting lug, each of the mounting lugs being structured to receive one of two ends of the connection band to connect and fix thereto.

11. The urine discharge device according to claim 8, further comprising a urine bag, the urine bag being connected to and in communication with the connecting section of the spreading piece.

12. The urine discharge device according to claim 8, further comprising a urine drain tube and a urine bag, the urine drain tube having an end connected to and in communication with the connecting section of the spreading piece and an opposite end connected to and in communication with the urine bag.
